(19) United States
(12) Patent Application Publication
(10) Pub. No.: US 2009/0099543 A1
(43) Pub. Date: Apr. 16, 2009

(54) Hands-free Medicine Vial Holder

(76) Inventor: Joseph Stein, Brooklyn, NY (US)

Correspondence Address: Joseph I. Stein, 1400 E 21 St., Brooklyn, NY, 11210, US

(21) Appl. No.: 11/872,647

(22) Filed: Oct. 15, 2007

(51) Int. Cl. A61B 19/00

(52) U.S. Cl. 604/403

(57) ABSTRACT

As an Orthopedist I have found that if I have to hold both the vial and the syringe while I draw up medications it poses numerous problems. The first problem is that when I was aiming the needle towards the small vial in my hand, if my aim were to be off there was a likelihood that I would end up sticking my hand. Another problem was that having only one free hand to draw up the syringe (while the other hand was holding the vial) required me to contort my fingers in such a way that led to fatigue. Another problem that came up was that in certain situations administering injections had to be done under aseptic conditions which became more difficult when I had to handle the vial at the same time. The Hands-free medicine vial holder solves all these problems.

### CROSS REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates to the healthcare field. It relates more specifically to the process of drawing up medication from sterile vials into syringe using hypodermic needles. Healthcare professionals drawing up medications from sterile vials into syringes using hypodermic needles are faced with the fact that a “third hand” to hold the vials would make life much easier. Although one can technically draw up a syringe with one hand (while holding the vial in the other hand) it is not easy and requires a bit of contortion of the fingers. In the course of a day a busy nurse will technically draw up tens of syringes of medications to administer. Repeatedly having to contort the fingers leads to fatigue of the fingers and hand. There are also situations where the drawing of medicine from vial has to be done under aseptic conditions using sterile gloves. If the nurse has to hold the vial also it increases the likelihood the he or she will contaminate their hands. Another problem is the fact when you are holding the vial in one hand and the syringe in the other hand and you are then aiming the needle towards the small vial in your hand, if your aim is off you end up sticking your hand. Not a very pleasant experience.

### BRIEF SUMMARY OF THE INVENTION

The Hands-Free Medicine Vial Holder allows one person to draw up medicines from a vial using sterile gloves without having to resort to a second person holding the vial and without having to contaminate his/her sterile gloved hand if the operator is working alone. The operator can maintain sterility by wearing sterile gloves. As the operator's hands are not in the path of the trajectory of the needle the risk of needle sticks are greatly reduced during the drawing-up phase. Also since the operator can use both hands to draw the syringe he does not have to contort his fingers so he avoids getting fatigue and cramps of the hand. The object of the invention is that the device acts as a “third hand” to allow the operator freedom to use both hands to quickly draw up a syringe.

### BRIEF DESCRIPTION OF THE SEVERAL VIEWS OF THE DRAWING

FIG. 1: This is a depiction of the front view of the device. It has two hollow round receptacles. To the left is the smaller hollow receptacle. To the right is the larger hollow round receptacle. They are attached from the rear by a rectangle shape base. Protruding from the left of the smaller receptacle and also from the right of the larger receptacle are screws that can be adjusted to hold vials in place inside the hollow receptacles

FIG. 2 This is a depiction of the right side of the device. To the left (rear) of the drawing is the smaller round hollow receptacle with a screw protruding from it and to the right (towards the front) is the larger hollow receptacle with a screw protruding from it. Both receptacles are mounted onto a base at a 45 degree angle. The base is consists of a strip of rectangular metal that has screws on each side (to mount the device to a fixed surface). Only the screw on the right is shown in this drawing. Extending from the bottom of the base is another strip of metal curved upwards at a 45 degree angle that the receptacles are attached to. Extending from the top of the base is another strip of metal that extends vertically making the base look like a hollow vertical triangle.

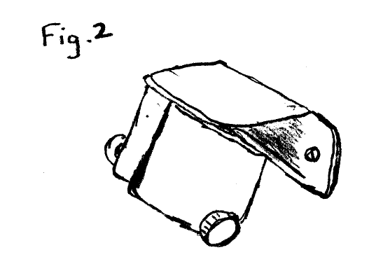

FIG. 3 This is a depiction of the left side of the device. To the left (front) of the drawing is the smaller round hollow receptacle with a screw protruding from it at the bottom and to the right (towards the rear) is the larger hollow receptacle. Both receptacles are mounted onto a base at a 45 degree angle. The base is consists of a strip of rectangular metal that has screws on each side (to mount the device to a fixed surface). Only the screw on the left of the base is shown in this drawing. Extending from the bottom of the base is another strip of metal curved upwards at a 45 degree angle that the receptacles are attached to. Extending from the top of the base is another strip of metal that extends vertically making the base look like a hollow vertical triangle.

FIG. 4 This is a depiction of the bottom view of the device. To the left is the smaller round hollow receptacle. To the right is the larger hollow round receptacle. From this angle you can see the inside the hollow portion of both receptacles. There are threaded screws protruding to the left of the smaller receptacle and to the right of the larger receptacle. Towards the top you can see part of the base that the receptacles are mounted to from the rear at a 45 degree angle. In the rear of the receptacles the rectangle shaped portion of the base that is mounted on to a fixed surface is visible. The screw on the left of the base is also visible.

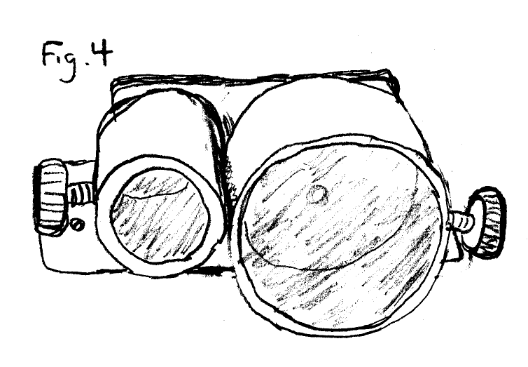

FIG. 5 This is a depiction of the top of the device. To the bottom left is the smaller round hollow receptacle. To the bottom right is the larger hollow round receptacle. Protruding from the left of the smaller receptacle and from the right of the larger receptacle are treaded screws used to keep the vials in place. To the top rear of the picture the rectangular portion of the base that is mounted onto a fixed surface is visible. The screws on both the left and the right side of the base used to mount it are also visible. The part of the base that extends outwards vertically is also visible on top of the receptacles.

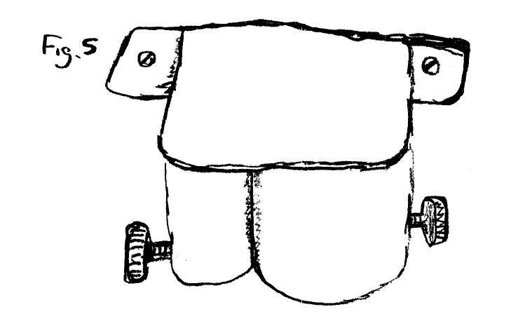

### DETAILED DESCRIPTION OF THE INVENTION

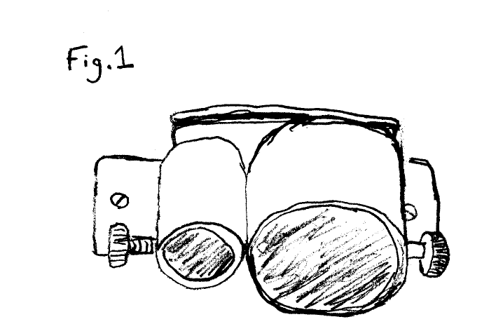

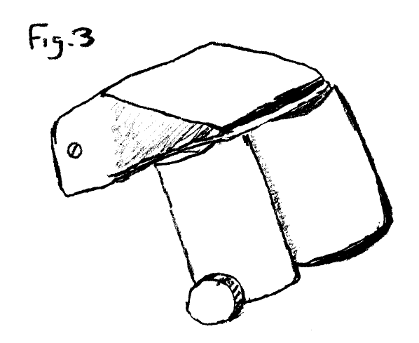

This section is has also been referred to as the “Description of the Preferred Embodiment”. The invention is referred to as the “Hands-free medicine vial holder”. It is used in the Healthcare field. It is used when healthcare professionals are drawing up medications from sterile vials into syringes using hypodermic needles. The hands-free medicine vial holder (“invention”) holds the vial in place and acts as a “third hand” to allow the operator freedom to use both hands to quickly draw up a syringe. The invention is made of machined and formed aluminum. It attaches to a fixed surface such as a nurses medication cart with screws. It has two round hollow receptacles to hold the most popular size medication vials. The larger receptacle which is located on the right in the drawing (in FIG. 1) holds 50 ml vials. The smaller receptacle located on the right of the drawing (in FIG. 1) holds 5-10 ml vials. Holders for other size vials can be custom ordered. The vials are held in place by set screws that can be adjusted to make sure the vials stay in place. The screws are threaded with a knurled knob heads and have a conical point. The receptacles are attached to a base and extend at a 45% angle towards the operator making it easy to draw up fluids. It is very simple to use. The base that the receptacles is affixed to consists of a rectangular shaped piece of aluminum with holes on the right edge and on the left edge so you can place screws in the holes to affix the invention to a fixed surface. Extending from the bottom of the base is a strip of aluminum bent upwards at a 45 degree angle which both receptacles are attached to. Extending vertically from the top of the base is another strip of aluminum that is mostly for aesthetic purposes. In FIG. 3 which shows the left side of the device the base appears to be a hollow triangle. There is a device on the market that is HANDHELD and not fixed to a surface that holds a vial at a distance from the operator's hand. This device is marketed to prevent needle sticks. But it does not provide a “third hand” for the operator as it must be held in one of the operators hands. The improvement of the Hands-free medicine vial holder over previous devices is that it is hands-free and it is affixed to a fixed surface. The improvement more specifically allows one to use both hands to draw up medication with the syringe. This ensures that their sterile gloved hands will remain sterile, being they don't have to hold the vial at the same time. It also ensures that the operators hands will not be fatigued being that if he would have to hold both the vial and the syringe at the same time he would have to contort his fingers in order to draw up the medication.

1. A medical device that holds vials in place comprising: a base with holes on the left and right edges to insert screws that will affix the device to a fixed surface, the base being a rectangular shape; a strip of aluminum extending from the bottom of the base upwards at a 45 degree angle; two round hollow receptacles of different sizes that are attached to the base at the part where the aluminum strip extends from the bottom of the base, the receptacles being tilted at a 45 degree angle; two threaded screws protruding from each receptacle, the screws having knurled knob heads and have conical points; a strip of aluminum extending from the top of the base vertically which can serve both to strengthen the device and can serve an aesthetic function
